(19) United States
(12) Patent Application Publication
(10) Pub. No.: US 2019/0255144 A1
(43) Pub. Date: Aug. 22, 2019

(54) SPECIFIC OLIGOPEPTIDES AS ANTI-ANGIOGENIC DRUGS

(71) Applicants: NEURONAX, Saint-beauzire (FR); UNIVERSITE DE LIMOGES, Limoges (FR)

(72) Inventors: Stephane GOBRON, Dallet (FR); Fabrice LALLOUE, Isle (FR); Romain BIBES, Limoges (FR); Francois VINCENT, Boisseuil (FR)

(21) Appl. No.: 15/749,020

(22) PCT Filed: Jul. 31, 2015
(86) PCT No.: PCT/FR2015/052133

§ 371 Date: Jan. 30, 2018

(30) Foreign Application Priority Data
Jul. 31, 2014 (FR) 1457407

(51) Int. Cl. A61K 38/17; A61P 35/00; A61K 45/06

(52) U.S. Cl. CPC A61K 38/1709; A61K 45/06; A61P 35/00

(57) ABSTRACT

An oligopeptide isolated from an SCO-spondin glycoprotein, characterized by the sequence: —W—S—X₁—W—S— X₂—C—S— X₃—X₄—C-G- (SEQ ID NO: 1), in which X₁, X₂, X₃ and X₄ represent amino acid sequences that consist of 1 to 5 amino acids. The oligopeptide may be used as a drug for inhibiting angiogenesis, in the treatment of diseases associated with angiogenesis in humans or animals, in particular against tumor angiogenesis. The oligopeptide is suitable in particular for the treatment of brain tumors.

### FIELD OF THE INVENTION

This invention relates to a specific oligopeptide and its use as a medication in the treatment of diseases associated with angiogenesis in humans or animals, in particular as an anti-tumor agent.

### BACKGROUND OF THE INVENTION

Angiogenesis is the mechanism that is responsible for the formation of new vessels from preexisting vessels. It is essential during certain physiological processes, in particular for the establishment of the vascular system in embryos and for the implanting of placentas, but it can also be responsible for pathological developments, such as rheumatoid arthritis, macular degeneration in adults, and primarily the growth of tumors and the development of metastases. It is actually well established that the development of an intratumoral or peritumoral vascularization is a key event for the growth of a tumor and for the metastatic dissemination by means of blood.

This is why, for several years, research has been conducted on molecules that have anti-angiogenic effects that can be used in cancer treatment.

Most of the current anti-angiogenic agents primarily target the VEGF (“Vascular Endothelial Growth Factor,” factors of vascular endothelial growth) cascade produced by the tumor cells and its bond with receptors, in particular by using blocking antibodies or inhibitors of receptors with tyrosine kinases involved in angiogenesis and tumor proliferation.

However, although the VEGF is recognized as a significant regulating factor in tumor growth and although it constitutes a preferred target for anti-cancer treatments, the development of new angiogenesis-inhibiting agents represents a significant challenge for increasing the number of therapeutic options. In addition, the identification of such agents that act on neo- or anti-angiogenesis means would represent a significant advance that can make it possible to develop a therapeutic arsenal so as to address numerous pathologies in which these angiogenesis phenomena occur (macular degeneration, for example).

If some of the developed angiogenesis inhibitors currently show effects on the control of the growth of tumors in association with conventional chemotherapy, they are not, however, effective enough, in particular for the treatment of solid tumors in a general way and tumors of the central nervous system more specifically. Actually, some of these molecules, like the antibodies, can be molecules that have a high molecular weight that prevents them from spreading over long distances in particular into a tumor mass or easily crossing the hematoencephalic barrier.

In addition, the existing anti-angiogenic molecules are synthetic proteins that are complex and expensive to manufacture. They should also for the most part be used at a high concentration and have important secondary effects like, for example, AVASTIN® (Bevacizumab). These secondary effects, sometimes with a fatal outcome, encountered with Bevacizumab, are in particular a leukopenia at the origin of infections as well as a risk of hemorrhage. Other ways of blocking the path of the VEGF (anti-VEGF antibodies, anti-VEGF-R, small PI3K blocking molecules, etc.) are furthermore being developed, but in the vast majority of cases, they have limitations in terms of effectiveness, specificity, and undesirable effects, with the latter able to apply to the cardiovascular system. The occurrences of arterial hypertension are thus generally observed along with the various anti-angiogenics and myocardial toxicities for some of them (Wu, S. et al. Lancet Oncol 2008; 9: 407-11; Nalluri, S. R., et al. JAMA 2008; 300 (19): 2277-85; Chu, T. F., et al. Lancet 2007; 370: 2011-19).

There is therefore a need for an anti-angiogenic agent that overcomes these numerous drawbacks.

### SUMMARY OF THE INVENTION

To respond to this, the invention proposes a specific oligopeptide whose sequence is derived from a portion of the TSR (“Thrombospondin type 1 repeat”) patterns that are present in the SCO-spondin, specific glycoprotein of the central nervous system and present in all of the vertebrates, from prochordals to humans. It is a molecule of extracellular matrices that is secreted by a specific organ located in the roof of the third ventricle, the sub-commissural organ. The SCO-spondin is a molecule of large size that consists of more than 4,500 amino acids and that has a multi-modular organization that comprises various preserved protein patterns, including in particular 26 TSR patterns. It is known that certain peptides derived from SCO-spondin starting from TSR patterns have a biological activity in the nerve cells (in particular described in the application WO-99/03890) and that these peptides act on the nerve cells by means of a specific receptor (M. Bambad et al., Cell & Tissue Research, Vol. 315, 15-25 (2004)).

The TSR patterns are protein domains of approximately 55 residues, based on the alignment of preserved amino acids cysteine, tryptophan and arginine. These patterns were first isolated in TSP-1 (thrombospondin 1) that is a molecule that intervenes in coagulation. They were then described in numerous other molecules with various biological functions such as cellular attachment, mobility, proliferation, cellular aggregation, modulation of proteases or inhibition of angiogenesis.

TSP-1 is a glycoprotein of the extracellular matrix with a molecular weight of approximately 120 kDa in its monomer form, having an area of bonding with heparin and repeated patterns of three types that can be bound to a certain number of angiogenic factors, including FGF-2, VEGF, PDGF and TGF-β1 (Lamszus et al., 1996; Margosio et al., 2003). This interaction makes possible the sequestration of these factors and limits their interactions with their respective receptors. The second mechanism of action of the TSP-1 passes in particular by its bond with endothelial cells via the proteoglycans, the integrins, CD47 and CD36 (Asch et al., 1987; Gao et al., 1996; Primo et al., 2005; Zhang et al., 2009b). The interaction of TSP-1 with CD36 would be able to inhibit the pro-angiogenic signaling by forming a complex with NRP-1 and VEGFR-2 that would prevent the activation of the VEGFR-2 induced by the VEGF. The TSP-1 can in addition act on the NOS, inhibit the migration of the endothelial cells induced by the FGF-2, and also induce the apoptosis of the endothelial cells. Furthermore, it was described that the anti-angiogenic properties of TSP1 involved an inhibition of the survival, proliferation, and migration of the endothelial cells (Jimenez, B., Volpert, O. V., Crawford, S. E., Febbraio, M., Silverstein, R. L., Bouck, N. Nat Med. 2000 Jan; 6(1): 41-8).

It is known in a general manner that certain peptide fragments of the TSP-1 as well as certain synthetic peptides whose structure is analogous to that of these peptide fragments show an activity that is similar to the native thrombospondin-1.

By way of example, the patents U.S. Pat. No. 6,239,110, U.S. Pat. No. 5,840,692, U.S. Pat. No. 5,849,701, U.S. Pat. No. 5,491,130 and U.S. Pat. No. 6,051,549, U.S. Pat. No. 5,190,918 and U.S. Pat. No. 5,200,397 and U.S. Pat. No. 6,384,189 describe the biological structure and effects of several of these peptide fragments that are derived from TSP-1 and from its synthetic analogs.

In addition, a certain number of molecules have been developed for anti-angiogenic purposes, inspired by the TSR patterns of TSP-1. Several peptide derivatives or peptides of this type, presented as being able to mimic the effects of TSP-1, in particular ABT-510 and ABT-526, are known. However, despite the close amino acid sequences and an affinity for their receptor, the solubility and the effect of these synthetic peptides on the endothelial cells can vary (Haviv et al., 2005). The ABT-510 in particular has proven itself to be an in-vitro inductor of apoptosis that depends on the Capsase-8 via CD36 and reduced the tumor vascularization in a preclinical model of glioma heterotopic grafting on nude mice (Anderson et al., 2007). Furthermore, it was the subject of clinical studies in various cancers, in particular sarcoma and renal carcinoma. However, although its harmlessness has been demonstrated, the monotherapy effectiveness of the compound ABT-510 was judged insufficient (Baker et al., 2008; Ebbinghaus et al., 2007).

Other peptide compounds have been developed from TSR patterns of the TSP-1 such as, for example, the 3TSR (Ren et al., 2009), or have been studied coming from other proteins that have TSR patterns, such as, for example, ADAMTS5, from which a recombinant protein bearing the TSR patterns was obtained (Sharghi-Namini et al., 2008). The mechanism of action of this recombinant protein remains VEGF-dependent, however. Actually, the recombinant protein showed that it inhibited the stimulation of the endothelial cells by VEGF and induced their apoptosis in the presence of VEGF. Furthermore, obtaining this recombinant protein in sufficient quantity makes it necessary to resort to expression systems that can be complex for a pharmaceutical quality because of being able to introduce contaminations or to modify the conformation (tertiary structure) of the protein that is to be produced.

Currently, the sequences of the TSR patterns that are known for their effectiveness are:
The sequence V-T-C-G (SEQ ID NO: 2) that can be bound to the receptor CD36 or gpIV of the platelets;
The sequence C—S—V-T-C-G (SEQ ID NO: 3): S. S. Tolsma et al. (The Journal of Cell Biology, Vol. 122 pp. 497-511 (1993)) showed that the anti-angiogenic activity of TSP-1 could be reproduced in vitro and in vivo with various synthetic peptides, including in particular two sequences that are derived from the TSR domain and that have the sequence C—S—V-T-C-G (SEQ ID No. 3) in the central position. In addition, subsequent studies have specified that the activity of neovascularization by TSP-1 could take place according to two different mechanisms that involved two different regions of the TSR, including a region that contains the sequence C—S—V-T-C-G (SEQ ID No. 3) (M. L. Iruela-Arispe, Circulation, Vol. 100, pp. 1423-31 (1999));
The sequence W—S—X—W (SEQ ID NO: 4) that is able to link heparin and proteoglycans that thus make possible a variable biological effect and that intervene in the process of activation of the TGFβ by the TSP-1. The presence of this W—S—X—W pattern (SEQ ID NO: 4), although not sufficient in itself, is necessary for this activation of the cytokine that can then take place in various biological events such as inflammation, angiogenesis or embryonic development (G. D. Young and J. E. Murphy-Ullrich, The Journal of Biological Chemistry, Vol. 279, pp. 47633-47642 (2004)). In addition, this pattern seems to act as a competitor of certain pro-angiogenic growth factors during their bonding with proteoglycans that are present on the surface of the endothelial cells (T. Vogel et al., The Journal of Cellular Biochemistry, Vol. 53, pp. 74-84 (1993)). It is also a matter of the second region of the TSR that is cited in M. L. Iruela-Arispe (Circulation, Vol. 100, pp. 1423-31 (1999)) that takes place in the mechanisms that are responsible for the neovascularization activity by the TSP-1. However, the exact composition of the variant amino acid (X) or additional amino acids located upstream or downstream from this pattern is important for the activity of the peptide. This is the case in particular of the effectiveness of the bond with heparin and the induction of the cellular adhesion that are increased for a pentapeptide where X is proline or basic amino acids (N. H. Guo, The Journal of Biological Chemistry, Vol. 267, pp. 19349-55 (1992)).

To date, no anti-angiogenic product that is effective enough and well-tolerated has been developed, and no product built on the basis of all of the elements described above has been tested for anti-angiogenic activities.

This is why the invention proposes an effective and well-tolerated specific oligopeptide, which eliminates certain drawbacks of the existing anti-angiogenic treatments.

In particular, the object of the invention is an oligopeptide that has the following sequence:

(SEQ ID NO: 1)
-W-S-X₁-W-S-X₂-C-S-X₃-X₄-C-G-

in which X₁, X₂, X₃ and X₄ represent amino acid sequences that consist of 1 to 5 amino acids, as an angiogenesis-inhibiting medication. It is useful in the treatment of diseases that are associated with angiogenesis, i.e., diseases characterized by an excessive or abnormal angiogenesis or by a proliferation of endothelial cells. This sequence of amino acids was described in its form —W—S-G-W—S—S—C—S—R—S—C-G- (SEQ ID NO: 5) in the patent WO9903890 and WO2009027350 but not at all for anti-angiogenic activities.

In a surprising and unexpected way, the oligopeptide according to the invention plays a role of protecting the apoptosis of the HUVECs or HBMECs, in a comparable way to a high dose of VEGF (50 ng/mL) and does not modulate the proliferation thereof. In vitro, the effects of the oligopeptide according to the invention on the endothelial cells therefore seem opposite to those of the TSP-1. Furthermore, the oligopeptide according to the invention promotes the migration in the presence of an angiogenic factor such as VEGF or bFGF. It inhibits, however, the migration of the HBMECs induced by glioblastoma tumor cells when it is brought into contact with the U87-MG or HBMECs.

In addition, it was demonstrated that in contrast to TSP-1, the action of the oligopeptide that is the object of this invention does not involve the receptor CD36.

It thus has an inhibitor role on the vascularization mechanisms and a significant anti-angiogenic effect that correlates the anti-tumor effect that is also observed. Because of its small size, it is also particularly suited to the treatment of tumors of the central nervous system, in particular glioblastomas.

The oligopeptide according to the invention for its use as an anti-angiogenic is unique because of its effects and its original mechanisms of action. Actually, ABT-510, which is the closest anti-angiogenic molecule because of its origin, derives from the central part of the second type-1 thrombospondin repetition that is located at the N-terminal end of TSP-1, protein expressed by the numerous cellular types and involved in various biological processes, including angiogenesis, whereas the oligopeptide according to the invention represents the consensus sequence of the N-terminal part of the 27 TSR repetitions of the SCO-spondin that is a protein that has TSR patterns but that is specifically expressed in the area of the SNC and intervenes in particular during development.

The oligopeptide has in addition improved safety, with the oligopeptide according to the invention showing no toxicity even with injections of 20 mg/kg.

### BRIEF DESCRIPTION OF THE DRAWINGS

Other characteristics and advantages will emerge from the following description of the invention, taking into account the accompanying drawings in which:

FIGS. 1A to 1D show macroscopic observations (20× magnification) produced after injection of aqueous fluorescein in the systemic circulation of an embryo at ED9:
FIG. 1A: water (control)
FIG. 1B: Bevacizumab (negative control)
FIG. 1C: VEGF with 10 ng/mL (positive control)
FIG. 1D: oligopeptide according to the invention, 250 μg/mL

FIGS. 2A to 2C represent analysis results of soluble factors that occur in angiogenesis:
Supernatants of U87-MG cells were studied by proteome array (FIGS. 2A and 2B) for the research of proteins involved in angiogenesis, in the presence of sterile water, TSP-1 and the oligopeptide according to the invention. The secretions of VEGFA via the U87-MG in the medium after treatments with sterile water, TSP-1 and the oligopeptide according to the invention, were metered by ELISA from supernatants (FIG. 2C).

FIGS. 3A to 3C show results of the analysis of the effect of the oligopeptide according to the invention on angiogenesis and the tumor proliferation in the model of the CAM:

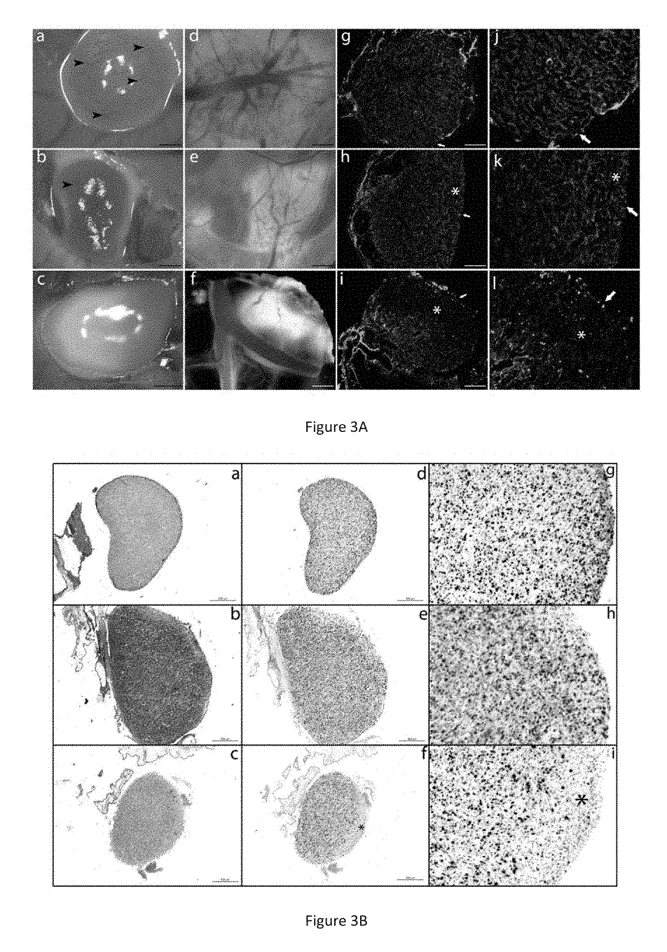

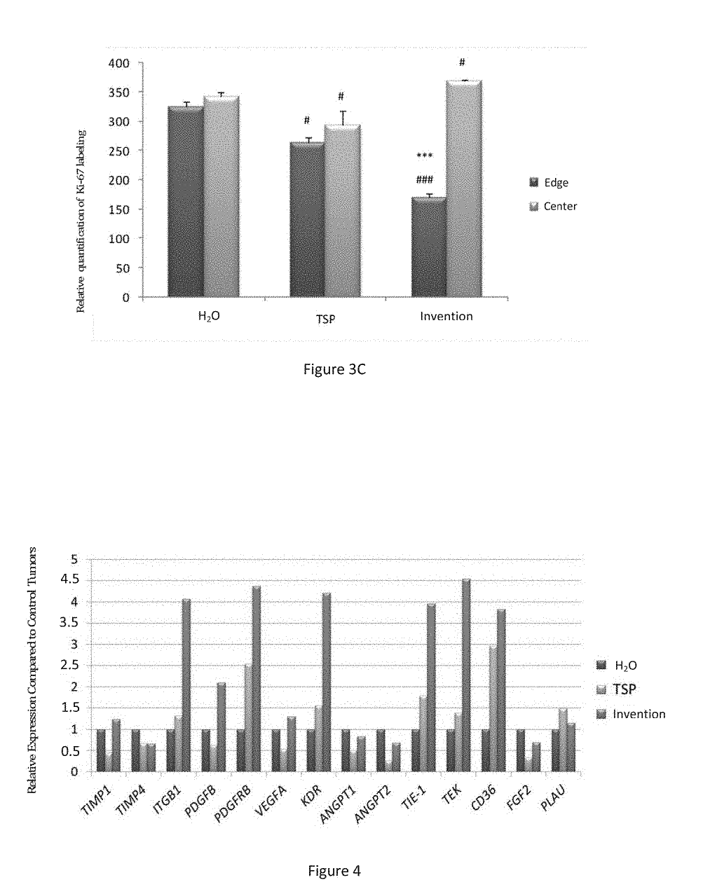

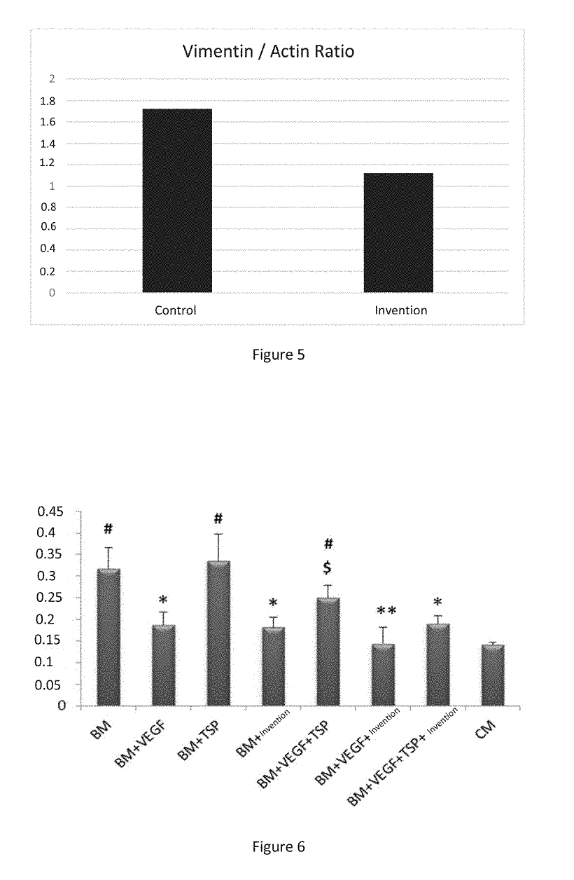

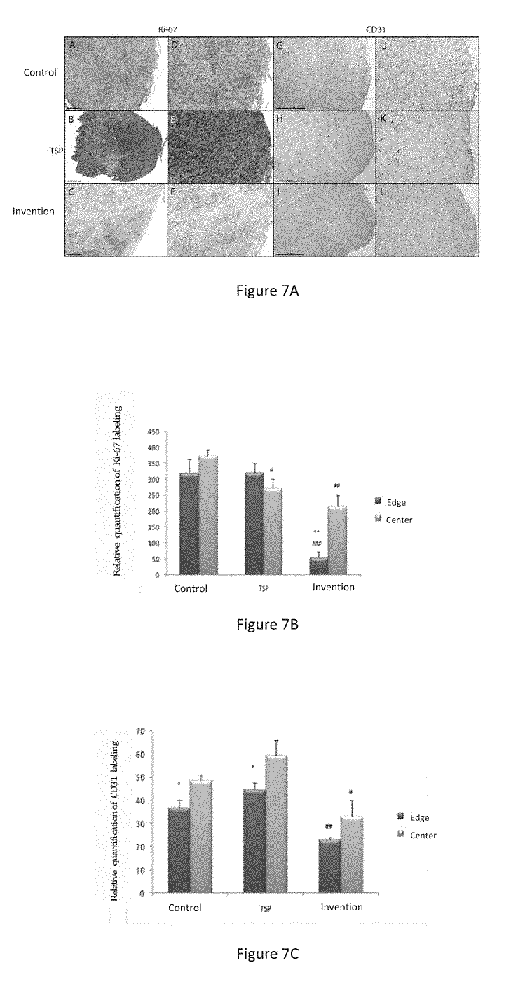

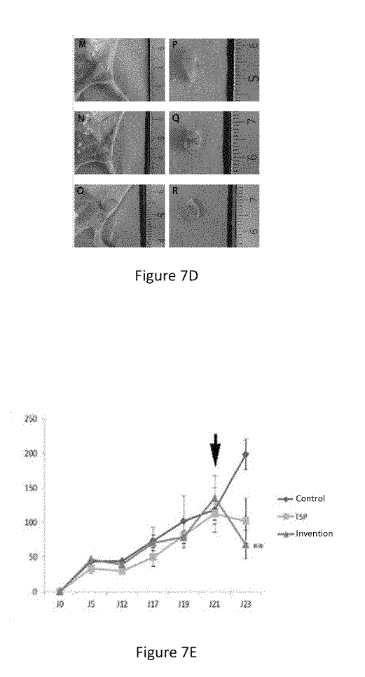

From tumors obtained from U87-MG grafts on the CAM, the vascularization of the experimental tumors have been studied on macroscopic levels (FIG. 3A—a to f) and microscopic levels (FIG. 3A—g to 1) after treatment for 48 hours with:
H₂O: a, d, g and j
TSP-1: b, e, h and k
Oligopeptide according to the invention: c, f, i and l

The microscopic observation of the vessels is carried out after labeling the former with SNA-1. The bars of the scale for the images h and i of FIG. 3A represent 1 mm, and the images j, k and l of FIG. 3A are magnifications (2×) of the images g, h and i of FIG. 3A, respectively; the heads of arrows show visible vessels, the arrows show the treated surfaces, and the stars show the zones without vessels.
The structure of the tumors was visualized after staining with hematoxylin, eosine, and safran from experimental tumor sections (FIG. 3B—a to c), and the proliferation of tumor cells was studied by immunohistochemical staining of Ki-67 with counter-staining by hematoxylin (FIG. 3B—d to i). The tumors were treated for 48 hours with
Water: a, d and g
TSP-1; b, e and h
The oligopeptide according to the invention: c, f and i

The images g, h and i of FIG. 3B are magnifications (4×) of the edges of the images d, e and f of FIG. 3B, respectively. The arrows show the treated surfaces, and the stars show the unmarked areas of Ki-67.
The Ki-67 labeling was quantified (FIG. 3C) in the various conditions using imageJ software, and comparisons between the center and the edge of tumors and between the conditions were carried out by analysis of variances (ANOVA). A value of p<0.05 is considered to be significant. * p<0.05; ** p<0.01; *** p<0.001 in comparison to the center of the same tumor. $ p<0.05; $$ p<0.01; $$$ p<0.001 in comparison to the control tumor.

FIG. 4 shows the results of the quantitative analysis of the mRNA in experimental tumors obtained from CAM, with the various genes being represented by their relative expression compared to the control tumors

FIG. 5 shows the results of the semi-quantitative analysis of the protein expression of the vimentin by Western Blot with or without the oligopeptide according to the invention on the U87-MG cells,

FIG. 6 shows the results of the analysis of the functional effect of the oligopeptide according to the invention on the apoptosis of HBMECs in vitro. It involves a synthesis of an analysis by ELISA Cell Death: the treatments by VEGF, TSP-1, and the oligopeptide according to the invention (NX) have been carried out separately or in combination in the basal medium (BM). The complete medium (CM) and the basal medium (BM) were used as controls. A value of p<0.05 is considered to be significant. The comparisons between various treatments were carried out by analysis of variances (ANOVA test).
* p<0.05; ** p<0.01 compared to BM; $ p<0.05 compared to BM+NX;
# p<0.05 compared to CM,

FIGS. 7A to 7E show the results of the analysis of the activity of the oligopeptide according to the invention on the proliferation, the vascularization, and the tumor growth in nude mice:
The images A, B and C of FIG. 7A show the immunolabeling of Ki-67 on tumor sections treated by H₂O, TSP-1 or the oligopeptide according to the invention respectively.
The images D, E and F of FIG. 7A are magnifications (2×) of FIGS. A, B and C.
The images G, H and I of FIG. 7A represent the immunolabeling of CD31 on tumor sections that are treated by H₂O, TSP-1 or the oligopeptide according to the invention respectively (the bars of the scale correspond to 500 μm).
The images J, K and L of FIG. 7A are magnifications (2×) of the images G, H and I.

The Ki-67 labeling was quantified under the various conditions using imageJ software, and comparisons between the center and the edge of tumors and between the conditions were carried out (FIG. 7B) by analysis of variances (ANOVA test). A value of p<0.05 is considered to be significant. * p<0.05 that is compared to the center of the same tumors; $ p<0.05 and $$ p<0.01 that is compared to the control.
The CD31 labeling was quantified under the various conditions using image J software, and comparisons between the center and the edge of tumors and between the conditions were carried out (FIG. 7C) by analysis of variances (ANOVA test). A value of p<0.05 is considered to be significant. * p<0.05 that is compared to the center of the same tumors; $ p<0.05 and $$ p<0.01 that is compared to the control.
The images M and P of FIG. 7D represent tumors of mice after treatment by hydrogel by itself.
The images N and Q of FIG. 7D represent tumors of mice after treatment by TSP-1 in hydrogel.
The images O and R of FIG. 7D represent tumors of mice after treatment by the oligopeptide according to the invention (NX) in hydrogel.
FIG. 7E shows the measurements of tumor growths that are treated with H₂O, TSP-1 and the oligopeptide according to the invention on 5, 12, 17, 19, 21 and 23 days after the injection of cancer cells. The injections of the treatments (hydrogel by itself, TSP-1 and the oligopeptide according to the invention (NX) were carried out on the 21^{st} day (black arrow)). The comparison of the tumor sizes in comparison to the controls was carried out by analysis of variances (ANOVA test). ** p<0.01; *** p<0.001.

### DETAILED DESCRIPTION OF THE INVENTION

The object of the invention is therefore an oligopeptide that has the following sequence:

(SEQ ID No. 1)
-W-S-X₁-W-S-X₂-C-S-X₃-X₄-C-G-

in which X₁, X₂, X₃ and X₄ represent amino acid sequences that consist of 1 to 5 amino acids, as an angiogenesis-inhibiting medication. It is useful in the treatment of at least one disease that is associated with angiogenesis, i.e., a disease that is characterized by an excessive or abnormal angiogenesis or by a proliferation of endothelial cells. Preferably, it is useful in the treatment of diseases that are associated with angiogenesis selected from among: cancers, arthritis, rheumatoid arthritis, atherosclerotic plaque, the neovascularization of a cornea graft, hypertrophic or keloid scars, proliferant retinopathy, diabetic retinopathy, macular degeneration in adults, granulation, neovascular glaucoma, and uveitis.

In terms of this invention, amino acids are defined as the natural amino acids and the non-natural amino acids.

“Natural amino acids” are defined as the L form of amino acids that can be found in proteins of natural origin, i.e.: alanine (A), arginine (R), asparagine (N), aspartic acid (D), cysteine (C), glutamine (Q), glutamic acid (E), glycine (G), histidine (H), isoleucine (I), leucine (L), lysine (K), methionine (M), phenylalanine (F), proline (P), serine (S), threonine (T), tryptophan (W), tyrosine (Y) and valine (V).

“Non-natural amino acids” are defined as the D form of natural amino acids, the homo forms of certain natural amino acids (such as: arginine, lysine, phenylalanine, and serine), and the “nor” forms of leucine and valine. They also comprise synthetic amino acids such as the alpha-aminobutyric acid (Abu), agmatine (Agm), alpha-arinoisobutyric acid (Aib), N-formyl-trp (F-trp), sarcosine, statin, ornithine, desaminotyrosine. The desaminotyrosine is incorporated at the N-terminal end whereas the agmatine and the statin are incorporated at the C-terminal end of these peptides.

In the sequence SEQ ID NO: 1, at least one amino acid can be replaced by one of its derivatives or the like.

According to a preferred embodiment, X₁, X₂, X₃ and X₄ each represent an amino acid.

Preferably, X₁ is selected from among G, V, S, P and A, X₂ is selected from among G, V, S, P and A, X₃ is selected from among R, A and V, and X₄ is selected from among S and P. The corresponding sequence is the sequence SEQ ID NO: 6: —W—S—X₁—W—S— X₂-C—S— X₃—X₄—C-G- in which X₁ is selected from among G, V, S, P and A, X₂ is selected from among G, V, S, P and A, X₃ is selected from among R, A and V, and X₄ is selected from among S and P.

According to a variant, X₁ and X₂ can be W—S—S—, W—S-G-, W—S—P—, W—S-A-, W—S—R—. The corresponding sequence is the sequence SEQ ID NO: 7: —W—S—X₁—W—S— X₂—C—S— —X₃—X₄—C-G- in which X₁ and X₂ are selected from among W—S—S—, W—S-G-, W—S—P—, W—S-A-, W—S—R—, and X₃ and X₄ represent the sequences of amino acids that consist of 1 to 5 amino acids.

The oligopeptide can be modified by amidation, acylation, pegylation, by addition of amino acids, but also by staple modification of peptides or by any method of coupling in particular with albumin or polymers such as PEG, or combination with a gel, hydrogel. The oligopeptide can also be cyclized.

The oligopeptide according to the invention can be obtained in various ways: by chemical synthesis in the liquid phase or on a solid substrate (Fmoc, for example), by other methods that resort to recombinant techniques and genetic designs comprising a DNA sequence or fragment that can code for peptide compounds that comprise the sequence of the oligopeptide according to the invention and that make it possible to collect the expression product by eukaryotic or prokaryotic expression systems or to express in situ the product. The oligopeptide according to the invention can come in deglycosylated form or else glycosylated form if this is necessary. In certain cases, and according to the preparation method, it may be necessary to renature certain tertiary structures of the oligopeptide.

The oligopeptide according to the invention:
Plays a role for protecting the apoptosis of the HUVECs or HBMECs and does not modulate their proliferation.
Promotes migration in the presence of an angiogenic factor such as VEGF or Bfgf.
Inhibits the migration of the HBMECs induced by glioblastoma tumor cells when it is put into contact with the U87-MG.

The oligopeptide according to the invention can therefore be used as an active ingredient for therapy or an anti-angiogenic medication in the treatment of various pathologies, in particular cancers, arthritis, rheumatoid arthritis, atherosclerotic plaque, the neovascularization of a cornea graft, hypertrophic or keloid scars, proliferant retinopathy, diabetic retinopathy, macular degeneration in adults, granulation, neovascular glaucoma, and uveitis.

Advantageously, it is capable of inhibiting the neo-angiogenesis of tumors in humans or animals.

According to another advantage, the small size of the oligopeptide of sequence SEQ ID NO: 1 facilitates the crossing of the hemato-encephalic barrier and the fast bio-distribution in the central nervous system as well as the targeting of the tumor zone. Its solubility in aqueous solutions makes it compatible with the clinical practice. It can therefore be used as a medication for the treatment of cerebral tumors in humans or animals, in particular for the treatment of glioblastomas.

Furthermore, since it is a matter of an oligopeptide that is obtained from a protein that is present in the organism, it has an increased tolerance and properties for inducing undesirable secondary effects that are restricted in relation to molecules that result from chemical synthesis or that are obtained from plants, as is the case of numerous current anti-angiogenics. In addition, it is soluble, non-toxic, and when it consists of natural amino acids, it is non-immunogenic and is easily eliminated by the organism.

The mode of action of the oligopeptide according to the invention is independent of CD36, in contrast to that of TSP-1. Actually, when the CD36 is activated by the TSP-1 in the endothelial cells, it interferes with the activation of the VEGF-R2 (KDR) and induces the apoptosis of the endothelial cells. The oligopeptide according to the invention promotes the drawing-together between the two receptors (CD36 and VEGFR-2), but increases the expression of CD36. Rather than play an inhibiting role directly on the endothelial cells, the oligopeptide according to the invention promotes the activation of VEGFR-2 in the presence of its ligand, with the path of the VEGF being known as promoting the survival of endothelial cells and their migration.

Furthermore, the oligopeptide according to the invention is capable of modulating two molecules: CD36 and VEGF-R2 that participate in the angiogenesis phenomenon.

The oligopeptide according to the invention can be administered by any suitable mode of administration. For the treatment of diseases that affect the central nervous system, it is preferably administered by intrathecal, intraventricular, intraparenchymal, or epidural means. For the other applications, it is preferably administered by intravenous means, by intraluminal means, by intravitreal means, by transphenoidal means, or by topical means, with these modes of administration not being limiting.

It can be integrated into pharmaceutical compositions in any form, in particular liquid or gel or any other substrate, in particular making possible controlled release.

The oligopeptide is preferably formulated in a pharmaceutical composition, in particular a pharmaceutical composition that comes in:
Liquid form: it can in particular be formulated in injectable preparations in the form of solutions, suspensions or emulsions (infusion preparations),
Or solid form, and it can comprise any type of substrate, in particular gels, biopolymers or biomaterials or any medical device such as implants or implantable pumps, for example, making possible a controlled release or else a vectorization system.

Within the framework of the treatment of tumors, the oligopeptide according to the invention can optionally be used by itself or in combination with one or more other anti-tumor active ingredients. It can be used with at least one anti-cancer active ingredient for chemotherapy, for example.

The object of the invention is therefore also the pharmaceutical compositions that comprise an oligopeptide that has the following sequence:

(SEQ ID NO: 1)
-W-S-X₁-W-S-X₂-C-S-X₃-X₄-C-G-

in which X₁, X₂, X₃ and X₄ represent amino acid sequences that consist of 1 to 5 amino acids, as described above for its use as an angiogenesis-inhibiting medication, with these compositions being useful in the treatment of diseases associated with angiogenesis as described above.

The invention is now illustrated by an example of an oligopeptide according to the invention and by test results demonstrating the anti-angiogenic effect of the oligopeptide according to the invention, in particular the oligopeptide of Example 1.

### EXAMPLE

### —W—S-G-W—S—S—C—S—R—S—C-G- (SEQ ID NO: 5)

This oligopeptide that is the object of this invention corresponds to the 12 most commonly preserved amino acids that are obtained from the fourth TSR domain of the SCO-spondin.

### Tests

A. Study of the Effects of the Oligopeptide According to the Invention on In-Vitro Angiogenesis

The apoptosis process is accompanied by the fragmentation of DNA and thus generates soluble nucleosomes (cytoplasmic histones associated with fragmented DNA), which can be metered by an immunoenzymatic method, ELISA Cell Death Plus (Enzyme-Linked ImmunoSorbent Assay) Kit (Roche Molecular Diagnostics, France) according to the recommendations of the supplier. The HBMEC cells were inoculated in a 24-well plate at the density of 5.10³ cells/wells and allowed to stand for 24 hours. The cells are cultivated in a basal medium, i.e., in the absence of growth factors, which is changed before they are exposed to various treatments for 24 hours. They are then recovered and then centrifuged with 200 g for 5 minutes. The cells are then counted, and then adjusted to 3.10⁴ cells/mL in PBS. A new centrifuging with 200 g is carried out for 10 minutes. The supernatant is removed, and the cell pellet is taken up in 200 μl of lysis buffer before incubation for 30 minutes at ambient temperature. After a new centrifuging for 10 minutes at 1,400 rpm, 20 μL of supernatant is distributed in wells coated with streptavidin and each condition is carried out in duplicate. 80 μL of the immunoreagent solution is added into each well. This solution contains the incubation buffer as well as 2 antibodies: an anti-histone antibody coupled to biotin and an anti-DNA antibody coupled to HRP (Horse Radish Peroxidase). The 2 antibodies will be linked to the plaque via streptavidin. The plaque is incubated for 2 hours at ambient temperature, while being stirred and in the dark. After this step, 3 washing cycles with 200 μL of incubation buffer are carried out so as to eliminate excess antibodies that are not attached. 100 μL of ABTS, which is the substrate of HRP, is then added into each well. The incubation with the ABTS solution is carried out in the dark for a period of 15 to 20 minutes. The reduction of the ABTS by the HRP makes possible the visualization of the apoptosis by colorimetric reaction. The reaction is then halted by adding 100 μL of ABTS stop solution, and the results are then analyzed using a 405-nm plaque reader. The number of oligonucleosomes that are formed is proportional to the intensity of the coloring and the cellular apoptosis.

The results that are obtained, presented in FIG. 6 and in Table 1 below:

TABLE 1
Mean Values Relative to the Optical Densities Measured for Each
Treatment Condition during the Test for Induction of the Apoptosis
on the HBMEC Cells

| Conditions | Optical Density Values | Mean Deviation |
|---|---|---|
| BM | 0.259 | |
| BM | 0.414 | |
| BM | 0.279 | 0.06444444 |
| BM + VEGF | 0.234 | |
| BM + VEGF | 0.133 | |
| BM + VEGF | 0.195 | 0.03622222 |
| BM + TSP | 0.215 | |
| BM + TSP | 0.419 | |
| BM + TSP | 0.372 | 0.08022222 |
| BM + NX | 0.199 | |
| BM + NX | 0.138 | |
| BM + NX | 0.21 | 0.02955556 |
| BM + VEGF + TSP | 0.214 | |
| BM + VEGF + TSP | 0.235 | |
| BM + VEGF + TSP | 0.304 | 0.03533333 |
| BM + VEGF + NX | 0.217 | |
| BM + VEGF + NX | 0.118 | |
| BM + VEGF + NX | 0.097 | 0.04866667 |
| BM + VEGF + TSP + NX | 0.212 | |
| BM + VEGF + TSP + NX | 0.206 | |
| BM + VEGF + TSP + NX | 0.154 | 0.02444444 |
| CM | 0.14 | |
| CM | 0.135 | |
| CM | 0.15 | 0.00555556 |

BM = Basal medium
CM = Complete medium
NX = Oligopeptide according to the invention

These results show that the oligopeptide according to the invention protects the HBMEC human endothelial cells from the apoptosis at a level similar to the VEGF, and this effect is not observed in the presence of TSP-1. In fact, TSP-1 is known for inducing the apoptosis of endothelial cells. Furthermore, the combined treatment of the oligopeptide according to the invention+VEGF improves this protective effect.
B. Study of the Effects of the Peptide According to the Invention on Angiogenesis Starting from a Chorioallantoic Membrane (CAM) Avian Model_{—} Angiogenesis is one of the key mechanisms of embryonic development. It is also a decisive process under certain physiological and pathological conditions (cancers in particular). This mechanism is controlled by numerous factors having either an inhibitor potential or an activator. So as to evaluate the main properties of these in vivo factors, the most common model is the CAM (chorioallantoic membrane) model from the chicken embryo (Ribatti et al., 2000). It offers the advantage of making it possible to test the pro- or anti-angiogenic factors during an active angiogenesis phase between the embryonic stages 4j and 10j. The major effects of the various molecules can therefore be evaluated in vivo. The model of the CAM can also be used in experimental tumor development. The grafting of human cancer cells onto the surface of the CAM is carried out in the 9-day embryonic stage. Three days after the grafting at ED12, the formation of a tumor is visible at the surface of the CAM. The latter can be treated topically during the 48 hours that follow this stage, and the tumors are sampled at ED14j.

### Detailed Protocol:

The fertilized eggs (Haas Poultry Farm, Strasbourg, France) are kept for 24 hours at 18° C. before the incubation is started up. They are then incubated in an incubator (by Maino, Italy), at 38° C. with a level of humidity of 45%. The embryonic stages are determined from tables of Hamburger and Hamilton (H and H, 1951). In the embryonic stage 2.5 j (ED 2.5) corresponding to stage 17 of H and H, 4 mL of albumin is removed using a 10-mL syringe, and then the contents of each egg are extracted from its shell and placed in hexagonal weighing dishes (VWR, Strasbourg, France). The latter are then placed in Petri dishes (VWR, Strasbourg, France) that contain water so as to ensure that a suitable hygrometry is maintained and to limit the risk of contamination, and then the dishes are set out to incubate. In this development stage, the chorioallantoic membrane begins to be vascularized. At stage ED9 (stage 35 of H and H), a silicone ring of approximately 1 cm in diameter and 1 mm in thickness is placed so as to surround several blood vessels. Two experimental approaches are carried out.

### 1) Treatments on the CAM

Various treatments (oligopeptide according to the invention, sterile distilled water, VEGF165, Bevacizumab) are then applied on the membrane using bovine gelatin-based resorbable sponges that are then deposited on the CAM (Bloxang, Chauvin Laboratory, France). The sponges are cut sterilely into cubes with 1-mm edges and are impregnated with the oligopeptide according to the invention at a concentration of 250 μg/mL. The impregnation volume produced is 50 μL per sponge. A control is carried out by impregnating the sponges with the same volume of sterile distilled water. Two additional controls of angiogenesis are deposited on the CAM, a sponge that is impregnated with VEGF165 at 10 ng/mL (positive control) and another impregnated with Bevacizumab at 250 μg/mL (negative control).

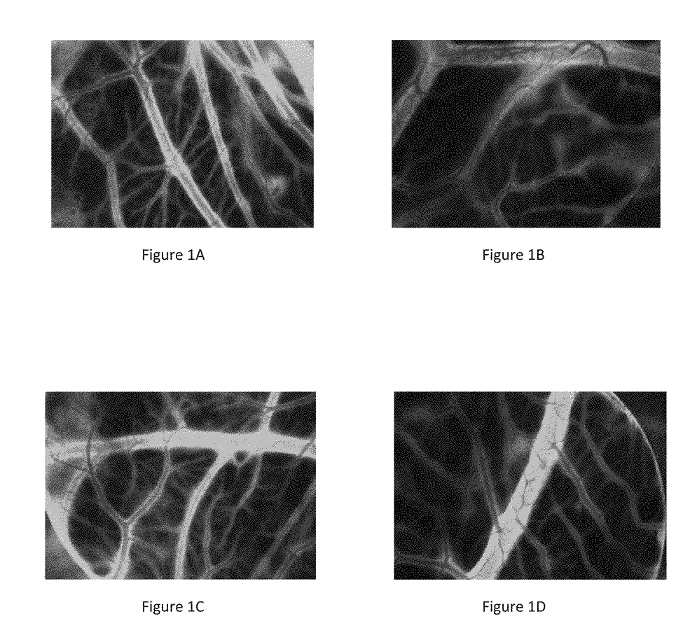

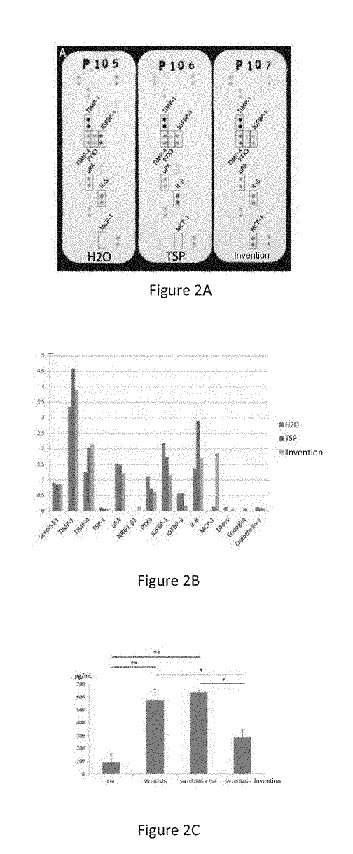

The results are presented in FIGS. 1A to 1D.

2) Treatments on the Tumors Obtained from U87-MG Grafting

To obtain a tumor, the U87-MG cells are inoculated at a rate of 200,000 cells in 75-cm² flasks in the presence of a basal medium. A volume that contains 4.10⁶ cells is then sampled, and after centrifuging for 5 minutes with 311 g, the supernatant is eliminated, and the cells are resuspended in 20 μL of differential medium that is deposited in the center of a silicone ring that was previously applied on the CAM of an embryo at ED9. Once the grafting is done, the embryo is set out to incubate. At ED11, the tumors can be observed under a MZFL3 binocular (Leica, Paris, France). At this stage, the tumors are either treated topically (apical part of the tumor) by 30 μL of the oligopeptide according to the invention at 250 μg/mL or else by 30 μL of sterile distilled water up to stage ED13. The tumors are then sampled, and cryosections are produced to make possible immunohistochemical analyses. The angiogenic network is displayed by injecting a 1% aqueous fluorescein solution (CHU, Limoges) into the vitelline artery using a micropipette. The macroscopic observations of tumors as well as the diffusion of fluorescein into the vessels are made with a fluorescence binocular (MZFL3, Leica, France).

The results that are obtained, presented in FIG. 1D, show an inhibition of vascularization, after 9 hours of treatment with the oligopeptide according to the invention at 250 μg/mL.

The experiment is carried out with injection of fluorescein isothiocyanate (FITC), which made it possible to facilitate the observation of the vascularization of the CAM on the angiography, and in particular deeper microvessels.

It is noted that during the treatment by the VEGF (FIG. 1C), a pro-angiogenic agent, the microvascularization is dense and comprises numerous vascular branches. In contrast, the anti-angiogenic effect of the oligopeptide according to the invention (FIG. 1D) is comparable to that of the Bevacizumab of the AVASTIN® (FIG. 1B) on the microvascularization. Its action seems particularly marked on the microvessels that constitute the vascular arborization while the largest vessels do not seem to be altered.
C. Study of the Effects of the Peptide According to the Invention on the Tumor Vascularization To obtain a tumor, the U87-MG cells are inoculated at a rate of 200,000 cells in 75-cm² flasks in the presence of a basal medium.

A volume that contains 4.10⁶ cells is then sampled and after centrifuging for 5 minutes with 300 g, the supernatant is eliminated, and the cells are resuspended in 20 μL of differential medium that is deposited in the center of a silicone ring that was previously applied on the CAM of an embryo at ED9. Once the grafting has been done, the embryo is set out to incubate.

At ED11, the tumors can be observed under a MZFL3 binocular (Leica, Paris, France), and it is possible to track the change in the tumor growth up to ED14.

The results are presented below:
a. Macroscopic Observation of the Effect of the Oligopeptide According to the Invention on the Tumor Angiogenesis in a CAM Model (FIGS. 3A to 3C)

The tumors that are developed on the CAM are observed on the chorioallantoic membrane using a MZFL3-type binocular (Leica, France) before being sampled.

Three days after the grafting of tumor cells, the tumor begins to develop, and vascularization begins. The pinkish color due to the presence of capillaries on the surface of the tumor disappears within the 24 hours following the beginning of the treatment by the peptide according to the invention on tumors that are more compact in size (1 to 2 mm in diameter) and between 24 and 48 hours for the tumors with diameters of greater than 2 mm. A pearlescent white color appears on the surface of the tumor, suggesting the establishment of a necrotic process that can lead to tumor regression (FIG. 3A c). The sampling of the tumor and of the CAM that is present in the ring (zone of treatment by the peptide according to the invention or the control) makes it possible to visualize the vessels that penetrate into the tumor.

A significant reduction in the density of the vessels that irrigate the tumor is noted following treatment with the oligopeptide according to the invention, thus confirming its anti-angiogenic activity suggested by the preceding results (FIG. 3A l).

The values that correspond to the results expressed in FIG. 3C are presented in Table 2 below:

TABLE 2
This table has the values of the quantification of the Ki-67 labeling
obtained for each treatment condition (TSP-1, oligopeptide according
to the invention NX and control without treatment Ctrl) based on the
tumor region being studied (edge and center).

| | Edge 1 | Edge 2 | Center 1 | Center 2 |
|---|---|---|---|---|
| CAM 1TSP1 | 227 | 314 | 300 | 292 |
| CAM 2TSP1 | 286 | 228 | 233 | 350 |
| CAM NX | 165 | 172 | 355.5 | 383.5 |
| CAM NX | 184 | 153 | 369 | 370 |
| CAM Ctrl | 310 | 342 | 331 | 346 |
| CAM Ctrl | 319 | 333 | 330 | 347 |

b. Study of the Vascularization and the Expression of Tumor Markers by Immunohistochemistry on the Tumor that is Treated with the Peptide According to the Invention (FIG. 5)

The vascular endothelium of the vessels of a chicken is labeled using a lectin, SNA-1 (Sambuccus Nigra Agglutinin-1, Vector Laboratories), coupled to FITC (fluorescein isothiocyanate).

After 10 minutes of attachment with 200 μL of 4% PBS 1X-PFA at ambient temperature, the sections are incubated with a solution of 0.05% TBS 1X-Tween-0.1% Trypsin, pH 7.6, for 10 minutes. They are then washed 3 times in PBS 1X, and 100 μL of a dilution with 1/200^{th} of the SNA-1 lectin in a solution of 0.05% TBS 1X-Tween with pH 7.6 is applied for 1 hour. After washing cycles (the last of which is carried out with distilled water), the slides are then mounted as described in the preceding paragraph and observed with a fluorescence microscope. Counts of vessels were made from photos with a magnification ×100 and in a marginal zone spreading 600 μm from the edge of the tumor. An ANOVA-type statistical test was then carried out to validate the relevance of the effect observed. The SNA-1 lectin specifically labels the chicken endothelial cells that constitute the vascular wall. The SNA-1 labeling on untreated tumor sections shows a richly vascularized structure with large vessels located at the base of the tumor. After treatment by the peptide according to the invention, the SNA-1 labeling disappears more specifically into the zone that is close to the surface of the tumor, suggesting a disappearance of the microvascularization in this region.

The vimentin/SNA-1 co-labeling made it possible to complete the preceding observations by providing the proof that the inhibiting of the tumor angiogenesis induced by the peptide according to the invention contributes not only to the regression of the tumor microvascularization but also the reduction in the number of differentiated tumor cells expressing the vimentin. The number of cells expressing the vimentin is larger in the control tumors than in the tumors (FIG. 5).
c. Study of the Cellular Proliferation after Treatment of the Tumors in the CAM Model by the Oligopeptide According to the Invention (FIGS. 7A to 7E)

The tumors are attached in 4% PBS 1X-PFA (24 hours) and then dehydrated in absolute ethanol and then in pure toluene and next are enclosed and preserved in a paraffin block. 4-μm sections are made from a microtome and deposited on Superfrost plus slides, and then dried in an oven at 50° C. for one night.

To carry out the immunolabeling, the sections are dewaxed and then rehydrated by baths of decreasing concentrations of ethanol and then rinsed in PBS 1X.

So as to unmask the antigenic sites, the slides are incubated in a citrate buffer with pH 7 (200 μM of citric acid; 9.8 mmol of sodium citrate) and undergo a heating cycle (4×5 minutes at 750 W in the microwave). The endogenic peroxidases are inhibited by carrying out a 10-minute bath in methanol-5%H₂O₂. After 3 washing cycles, the saturation of PBS-3% BSA and then the incubation with the anti-Ki67 antibody are carried out. After rinsing with PBS 1X, an HRP-conjugated secondary antibody is applied for 1 hour at ambient temperature. The revealing is carried out with DAB (3,3-diaminobenzidine). A counter-staining is carried out in hematoxylin, and the sections are mounted in an aqueous solution before being observed with a Leica DMRX microscope.

The labeling of the Ki-67 is distributed homogeneously over the entire control tumor (FIG. 7A D) while in the tumor that is treated by the peptide according to the invention (FIG. 7A F), the peripheral zone does not express the Ki67 marker. This region that exhibits a low Ki67 labeling corresponds to the band that is characterized above in which a very weak expression of vimentin and a significant reduction in the number of vessels are noted. This result therefore suggests a potential effect of the peptide according to the invention on the tumor cell proliferation that could be due to the significant reduction of the vascularization in the corresponding region.

The action of the peptide according to the invention on the inhibition of the tumor angiogenesis is therefore accompanied by a reduction in the expression of a marker of the tumor cells (vimentin) that is demonstrated using experimental tumors obtained on the CAM as well as a significant reduction of the proliferation of tumor cells.
d. Protein and Transcriptomic Analysis Conducted on Tumors that are Obtained on a CAM Model and Analysis of Variations of Secretion of Soluble Factors that are Involved in Angiogenesis Following the Treatment by the Oligopeptide According to the Invention

The secretion profiles of the cells show a significant increase in IL-8 in the medium of the cells that are treated by TSP-1, whereas the oligopeptide according to the invention induces a large increase in MCP-1 (FIGS. 2A and 2B) and a reduction in VEGF-A in the supernatant of U87-MG (FIG. 2C). The peptide therefore duly has an anti-angiogenic effect within a context that combines endothelial cells and tumor cells.

The values that correspond to the results that are expressed in FIGS. 2B and 2C are presented in Tables 3 and 4 below:

TABLE 3
Values relative to the results of the proteome array analysis of the
supernatants of U87-MG after treatment by H₂O (H2O), TSP-1 (TSP) or
oligopeptide according to the invention (NX) that is presented in FIG. B

| | H2O | TSP | NX |
|---|---|---|---|
| Serpin E1/Pal-1 | 0.92493716 | 0.85495271 | 0.86316564 |
| TIMP-1 | 3.34398164 | 4.59322792 | 3.88752813 |
| TIMP-4 | 1.24938431 | 2.0409603 | 2.15020158 |
| TSP-1 | 0.12252001 | 0.08481339 | 0.07983378 |
| uPA | 1.52097324 | 1.48877709 | 1.20853125 |
| VEGF | 0.77683673 | 1.39678618 | 1.02082894 |
| NRG1-β1/HRG1-β1 | 0 | 0 | 0.14646286 |
| Pentraxin 3 (PTX3)/TSG-14 | 1.09932408 | 0.71177425 | 0.61790135 |
| IGFBP-1 | 2.17561046 | 1.7235348 | 1.1652976 |
| IGFBP-3 | 0.56194267 | 0.57127158 | 0.18556444 |
| IL-8/CXCL8 | 1.38054145 | 2.90071833 | 1.69798791 |
| MCP-1/CCL2 | 0 | 0.15337442 | 1.86082798 |
| DPPIV/CD 26 | 0.1392225 | 0 | 0.08221834 |
| Endoglin/CD105 | 0 | 0.09235922 | 0 |
| Endothelin-1 (ET-1) | 0.12820021 | 0.09940002 | 0.09417925 |

TABLE 4
Values relative to the histogram showing the metering of VEGF in the
supernatant of U87-MG after treatment by H₂O (H2O), TSP-1 (TSP) or oligopeptide
according to the invention (NX) by ELISA that is presented in FIG. C

| | | | | | | Standard | | Mean |
|---|---|---|---|---|---|---|---|---|
| | Mean | Val1 | Val2 | pg/mL1 | pg/mL2 | Deviation pg/ml | std err | pg/mL |
| CM | 0.181 | 0.22 | 0.142 | 154.5 | 24.5 | 91.92388155 | 65.1942422 | 89.5 |
| SN U87 | 0.4745 | 0.521 | 0.428 | 656.1666667 | 501.1666667 | 109.6015511 | 77.7315965 | 578.666667 |
| SN U87 + | 0.509 | 0.5 | 0.518 | 621.1666667 | 651.1666667 | 21.21320344 | 15.0448251 | 636.166667 |
| TSP | | | | | | | | |
| SN U87 + | 0.2995 | 0.333 | 0.266 | 342.8333333 | 231.1666667 | 78.96025723 | 56.0001824 | 287 |
| NX | | | | | | | | |

Relative to the direct inhibiting action on the vessels of the oligopeptide according to the invention, it was shown that TSP-1 produces the same effects but to a lesser degree on this model of experimental tumors obtained from the CAM (FIGS. 2A, 2B and 2C). The oligopeptide according to the invention is therefore more effective than the TSP-1 in the inhibition of the tumor vascularization.

The study of the expression of genes in experimental tumors obtained from the CAM revealed overall an increase by a factor 4 in relation to the control of all of the genes coding for the receptors (ITGB1, PDGFRβ, KDR, TIE-1, TIE-2, CD3 6) that are being studied in the tumors that are treated by the oligopeptide according to the invention. These receptors belong to the chicken species and are therefore found in the host vessels. This increase in the expression of genes is also found in the tumors that have received the TSP-1 but on a lesser scale (FIG. 4). Furthermore, the overexpression of ITGB1 at the same time as that of CD36 differentiates the peptide according to the invention (NX) from the TSP-1 (TSP). In addition, the observed expression levels of PDGFRB, TIE1, TEK and KDR in the vessels of the experimental tumors nevertheless show that the oligopeptide according to the invention has a significant impact in the area of the tumor micro-environment (FIG. 4).

The values that correspond to the results expressed in FIG. 4 are presented in Table 5 below:

TABLE 5
The table shows the mean RQ obtained for each gene according to the
various treatment conditions. The mean RQ is obtained from a triplicate:
“con” treatments for control, “TSP” the TSP-1 treatment with the
thrombospondin 1 and NX treatment with the peptide according to
the invention.

| | Con | Mean RQ TSP | Mean RQ NX |
|---|---|---|---|
| TIMP1 | 1 | 0.44922254 | 1.23669395 |
| TIMP4 | 1 | 0.6642234 | 0.65521259 |
| ITGB1 | 1 | 1.31677272 | 4.06957648 |
| PDGFB | 1 | 0.63265723 | 2.10425551 |
| PDGFRB | 1 | 2.5435188 | 4.36553671 |
| VEGFA | 1 | 0.53918053 | 1.29771382 |
| KDR | 1 | 1.55729461 | 4.20663176 |
| ANGPT1 | 1 | 0.51056649 | 0.82414821 |
| ANGPT2 | 1 | 0.2678366 | 0.67868709 |
| TIE-1 | 1 | 1.79031205 | 3.96121105 |
| TEK | 1 | 1.38530537 | 4.53929902 |
| CD36 | 1 | 2.96481544 | 3.83116437 |
| FGF2 | 1 | 0.3429306 | 0.68597025 |
| PLAU | 1 | 1.49099798 | 1.15119235 |

D. Study of the In-Vivo Anti-Angiogenic Effect of the Peptide According to the Invention

The experimentation on the mouse confirms the results that are obtained with the CAM model. In this case, the tumors were obtained from subcutaneous heterotopic grafts in the area of the leg of a nude mouse (FIGS. 7B, 7C and 7E).

The values that correspond to the results expressed in FIGS. 7B, C and E are presented in Tables 6, 7 and 8 below:

TABLE 6

| | Edge1 | Edge2 | Center1 | Center2 |
|---|---|---|---|---|
| con | 451 | 187 | 440 | 322 |
| con | 288 | 368 | 396 | 397 |
| con | 218 | 402 | 322 | 364 |
| con | 37 | 31 | 46 | 51 |
| NX | 49 | 50 | 227 | 240 |
| NX | 52 | 44 | 280 | 290 |
| NX | 27 | 4 | 123 | 238 |
| NX | 75 | 163 | 264 | 89 |
| NX | 21 | 22 | 26 | 40 |
| TSP | 302 | 246 | 297 | 282 |
| TSP | 362 | 370 | 322 | 182 |
| TSP | 53 | 42 | 66 | 53 |

| | Mean Edge | Mean Center |
|---|---|---|
| Ctrl | 247.75 | 292.25 |
| TSP | 228.166667 | 200.333333 |
| NX | 49.75 | 181.7 |

TABLE 7

| | | | | | | | | Cen- |
|---|---|---|---|---|---|---|---|---|
| | Edge | Edge | Edge | Edge | Center | Center | Center | ter |
| CTRL | 37 | 31 | 33 | 46 | 46 | 51 | 42 | 55 |
| TSP1 | 53 | 42 | 39 | 45 | 66 | 53 | 63 | 56 |
| NX | 2′ | 22 | 26 | 23 | 26 | 40 | 28 | 38 |

TABLE 8

| Volume (mm³) | HydroGel | TSP | NX |
|---|---|---|---|
| D0 | 0 | 0 | 0 |
| D5 | 43.5866889 | 33.5535167 | 46.4597889 |
| D12 | 43.5633133 | 29.9250722 | 39.0419645 |
| D17 | 73.3539325 | 49.8769811 | 70.5983644 |
| D19 | 101.617334 | 81.0688689 | 78.82028 |
| D21 | 118.098889 | 112.518586 | 135.630556 |
| D23 | 197.82 | 101.875556 | 68.0333333 |

Tables 6, 7 and 8: These tables correspond to the raw values that are obtained during various analyses conducted on the tumors that are treated in vivo. Analysis of the proliferation (Ki67 labeling quantification) (F), analysis of the vascularization (CD31 labeling quantification) (G) and analysis of the tumor growth as a function of time (measurement of the tumor volume) (H). “con,” “CTRL” and “hydrogel” treatments are control conditions, “TSP” and “TSP1” are the treatments with thrombospondin 1 (TSP-1), and NX is the treatment with the peptide according to the invention

The tumors that are treated by the oligopeptide according to the invention decrease in volume 48 hours after the treatment and seem to be less vascularized than the control tumors and those that are treated by the TSP-1 (FIG. 7D R). The histological and immuno-histochemical studies have shown a reduction in the CD31 labeling on the periphery, by comparison with the control (FIG. 7A L, and FIG. 7C). This reduction in the number of vessels is accompanied by a reduction in cell proliferation, visualized by a decrease in the Ki-67 labeling (FIG. 7B).

The inhibition that is induced on the vascularization mechanisms by the oligopeptide according to the invention leads to the formation of a necrotic zone and the reduction in the expression of markers of the tumor cells that are not differentiated in the tumors that are treated in relation to the control tumors.

The oligopeptide according to the invention can therefore be used in particular for the treatment of diseases associated with angiogenesis, in particular glioblastoma, in particular within the framework of a multiple targeting.

1. A pharmaceutical composition comprising an oligopeptide of the sequence:
(SEQ ID NO: 1)
W-S-X₁-W-S-X₂-C-S-X₃-X₄-C-G
in which X₁, X₂, X₃ and X₄ represent amino acid sequences that consist of 1 to 5 amino acids, for use in humans or animals as an angiogenesis-inhibiting medication.

2. A method of treating of at least one disease associated with angiogenesis characterized by excessive or abnormal angiogenesis or by a proliferation of endothelial cells, comprising administering to a human or an animal in need there of an oligopeptide of the following sequence:
(SEQ ID NO: 1)
W-S-X₁-W-S-X₂-C-S-X₃-X₄-C-G
in which X₁, X₂, X₃ and X₄ represent amino acid sequences that consist of 1 to 5 amino acids.

3. The method according to claim 2, wherein the disease that is associated with angiogenesis is selected from the group consisting of cancers, arthritis, rheumatoid arthritis, atherosclerotic plaque, neovascularization of a cornea graft, hypertrophic or keloid scars, proliferant retinopathy, diabetic retinopathy, macular degeneration in adults, granulation, neovascular glaucoma, and uveitis.

4. The method according to claim 2, wherein X₁, X₂, X₃ and X₄ each represent an amino acid, with said amino acid being natural or non-natural.

5. The method according to claim 2, wherein X₁ is selected from the group consisting of G, V, S, P and A.

6. The method according to claim 2, wherein X₂ is selected from the group consisting of G, V, S, P and A.

7. The method according to claim 2, wherein X₃ is selected from the group consisting of R, A and V.

8. The method according to claim 2, wherein X₄ is selected from the group consisting of S and P.

9. The method according to claim 2, wherein said oligopeptide is modified by amidation, acylation, pegylation.

10. The method according to claim 2, wherein said oligopeptide is of the following sequence:
(SEQ ID NO: 5)
W-S-G-W-S-S-C-S-R-S-C-G.

11. The method according to claim 2, wherein the method treats cancers, and said oligopeptide inhibits neo-angiogenesis of tumors in said humans or said animals.

12. The method according to claim 2, wherein the method treats solid tumors that are tumors selected from group consisting of breast, lung, kidneys, intestine, colon, ovaries, and prostate.

13. The method according to claim 2, wherein the method treats cerebral tumors in humans or animals.

14. The method according to claim 2, wherein the method treats glioblastomas.

15. The method according to claim 2, wherein said oligopeptide is administered in association with at least one other anti-tumor active ingredient.

16. The method according to claim 2, wherein said oligopeptide is administered in association with an anti-cancer active ingredient for chemotherapy.

17. The method according to claim 2, wherein administrating said oligopeptide is carried out in association with an anti-cancer radiotherapy treatment.

18. The method according to claim 2, wherein said oligopeptide is administered by intrathecal, intraventricular, intraparenchymal, intravenous, subcutaneous, transphenoidal or topical administration.

19. The method according to claim 2, wherein said oligopeptide is administered within a pharmaceutical composition.

20. The method according to claim 19, wherein said oligopeptide is administered within a pharmaceutical composition that comes in the form of a solution, emulsion, suspension, gel, biopolymer or biomaterials.


SEQUENCE LISTING (7 sequences)

SEQ ID NO: 1 - protein, 28 aa, Artificial Sequence
  Peptide derived from SCO-spondine
  MISC_FEATURE (3)..(7): Xaa is any amino acid
  MISC_FEATURE (4)..(7): 1 to 4 of these amino acids may be absent
  MISC_FEATURE (10)..(14): Xaa is any amino acid
  MISC_FEATURE (11)..(14): 1 to 4 of these amino acids may be absent
  MISC_FEATURE (17)..(21): Xaa is any amino acid; 1 to 4 amino acids may be absent
  MISC_FEATURE (22)..(26): Xaa is any amino acid; 1 to 4 amino acids may be absent
WSXXXXXWSXXXXXCSXXXXXXXXXXCG

SEQ ID NO: 2 - protein, 4 aa, Artificial Sequence
  Peptide derived from TSR
VTCG

SEQ ID NO: 3 - protein, 6 aa, Artificial Sequence
  Peptide derived from   TSR
CSVTCG

SEQ ID NO: 4 - protein, 4 aa, Artificial Sequence
  Peptide derived from   TSR
  MISC_FEATURE (3)..(3): Xaa is any amino acid
WSXW

SEQ ID NO: 5 - protein, 12 aa, Artificial Sequence
  Oligopeptide derived from SCO-Spondin
WSGWSSCSRSCG

SEQ ID NO: 6 - protein, 12 aa, Artificial Sequence
  Oligopeptide derived from SCO-Spondin
  MISC_FEATURE (3)..(3): Xaa is Gly or Val or Ser or Pro or Ala
  MISC_FEATURE (6)..(6): Xaa is Gly or Val or Ser or Pro or Ala
  MISC_FEATURE (9)..(9): Xaa is Arg or Ala or Val
  MISC_FEATURE (10)..(10): Xaa is Ser or Pro
WSXWSXCSXXCG

SEQ ID NO: 7 - protein, 24 aa, Artificial Sequence
  Oligopeptide derived from   SCO-Spondin
  MISC_FEATURE (3)..(3): Xaa is Trp
  MISC_FEATURE (4)..(4): Xaa is Ser
  MISC_FEATURE (5)..(5): Xaa is Ser or Gly or Pro or Ala or Arg
  MISC_FEATURE (8)..(8): Xaa is Trp
  MISC_FEATURE (9)..(9): Xaa is Ser
  MISC_FEATURE (10)..(10): Xaa is Ser or Gly or Pro or Ala or Arg
  MISC_FEATURE (13)..(17): Xaa is any amino acid; 1 to 4 amino acids may be absent
  MISC_FEATURE (18)..(22): Xaa is any amino acid; 1 to 4 amino acids may be absent
WSXXXWSXXXCSXXXXXXXXXXCG